(19) United States
(12) Patent Application Publication
(10) Pub. No.: US 2023/0138347 A1
(43) Pub. Date: May 4, 2023

(54) SYNTHETIC SURGICAL HEMOSTAT

(71) Applicants: BAXTER INTERNATIONAL INC., Deerfield, IL (US); BAXTER HEALTHCARE SA, Glattpark (Opfikon) (CH)

(72) Inventor: Prasad Dande, Grayslake, IL (US)

(21) Appl. No.: 17/979,134

(22) Filed: Nov. 2, 2022

Related U.S. Application Data
(60) Provisional application No. 63/275,209, filed on Nov. 3, 2021.

(51) Int. Cl. A61L 26/00; C08L 89/06

(52) U.S. Cl. CPC A61L 26/0038; C08L 89/06; A61L 2400/04; C08L 2201/06; C08L 2203/02

(57) ABSTRACT

Disclosed herein are hemostatic materials and methods of production and use thereof.

### FIELD

The present Specification relates to the production and use of hemostatic materials.

### BACKGROUND

The rapid control of topical bleeding is of critical importance in wound management, particularly for the management of trauma, e.g., as a result of traumatic injury or surgery. Typical methods of controlling bleeding employ the use of “passive” devices including cotton gauze pads. Passive devices, however, do not initiate or accelerate blood clotting.

In contrast to passive devices, hemostats are “active” substances that promote hemostasis through the use of hemostatic agents, for example, fibrinogen or thrombin, and actively participate in the coagulation cascade to form a fibrin clot. Thrombin is a serine protease that plays important roles in blood clotting (coagulation). As the key coagulation protease, thrombin converts soluble fibrinogen into fibrin networks crosslinked by a transglutaminase (FXIII). In addition, thrombin is the most potent activator of platelets by stimulating protease-activated receptors (PAR). Upon activation by thrombin, platelets physically alter the conformation of GP IIb/IIIa receptors and provide high-affinity binding sites for fibrinogen, providing fibrinogen-crosslinked platelet aggregation.

However, the use of biologic clotting factors such as thrombin in hemostat formulations can raise costs and invite further complications such as supply chain impacts. Therefore, improved systems, devices, and methods are desirable.

### SUMMARY

The instant disclosure provides a novel class of hemostat materials with excellent mechanical properties, high fluid uptake, and biocompatibility, and methods of manufacturing thereof, for use in methods for establishing local hemostasis. Disclosed embodiments can also provide an antimicrobial effect. For example, disclosed methods can be used to prevent, limit, or reduce antimicrobial activity.

Disclosed hemostat materials and devices utilize synthetic biopolymers that can replace biologics such as thrombin in hemostat formulations.

Disclosed embodiments comprise particles comprising a cross-linked protein matrix and a synthetic biopolymer, for example feracrylum.

In embodiments, the hemostatic material is provided in a flowable state wherein it is able to “soak up” liquid material, such as blood.

Disclosed embodiments comprise methods of use. For example, disclosed methods and devices can be used to reduce or stop bleeding, for example bleeding associated with surgical procedures, injuries, wounds, and the like. Embodiments can comprise treatment of various categories of bleeding, including:

Grade 1: Mild
a. For example, capsular Liver Abrasion. Grade 1 Bleeds represent a general ooze, which well up over 1-2 minutes after blotting with gauze.

Grade 2: Moderate
a. Grade 2 bleeds visibly well up after blotting, and are usually considered distracting to the surgical procedure.

Grade 3: Severe
a. For example, rupture of venous plexus during posterior lumbar laminectomy. Grade 3 bleeds well up immediately after blotting, and require treatment to continue with the surgery.

Grade 4: Life Threatening a. For example, Abdominal Aortic Tear. Grade 4 bleeds are life-threatening and require immediate surgical treatment.

### BRIEF DESCRIPTION OF THE DRAWINGS

FIG. 1 shows (in triplicate) the thromboelastography (TEG; a hemostatic test that measures the shear elasticity and dynamics of dot formation, and the strength and stability of formed dot) profile for a 10% solution of the feracrylum in saline. The polymer alone has weak hemostatic properties. The colors represent the experiment in triplicate.

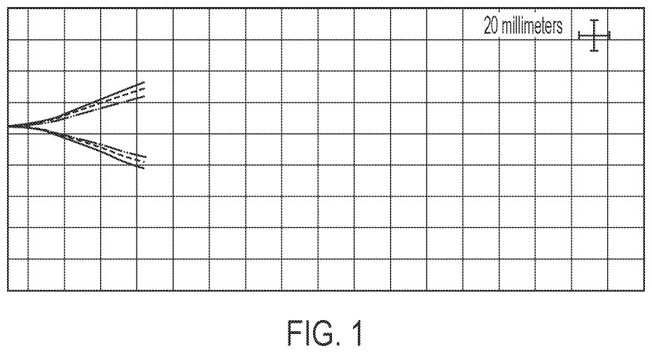

FIG. 2 shows (in triplicate) that the TEG profile for FLOSEAL crosslinked gelatin matrix reconstituted with a disclosed feracrylum formulation is an effective hemostat and results in rapid and strong clot formation as indicated by the curves. The steeper angle show a quicker clotting time. This curve compares favorably with the TEG profile obtained for FLOSEAL matrix reconstituted with thrombin solution per current FLOSEAL IFU depicted in FIG. 3.

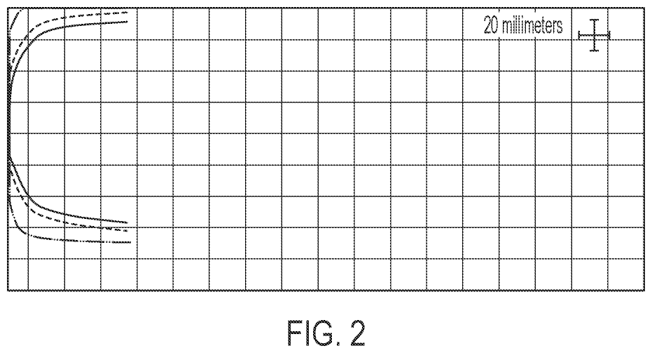

FIG. 3 shows FLOSEAL matrix reconstituted with thrombin solution per current FLOSEAL IFU. As compared to FIG. 2, FIG. 2 shows quicker clotting time.

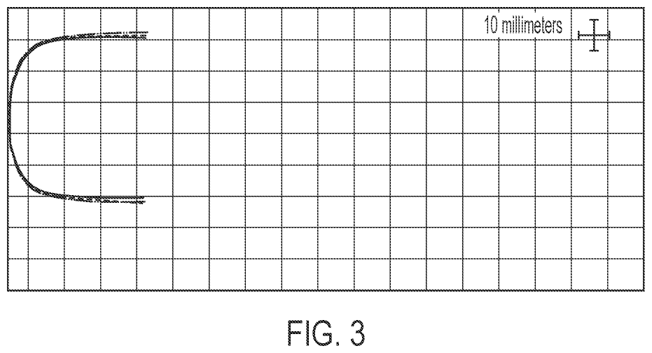

FIG. 4 shows that FLOSEAL matrix reconstituted without coagulation enhancer (i.e. reconstituted with 0.9% saline) has no coagulation activity.

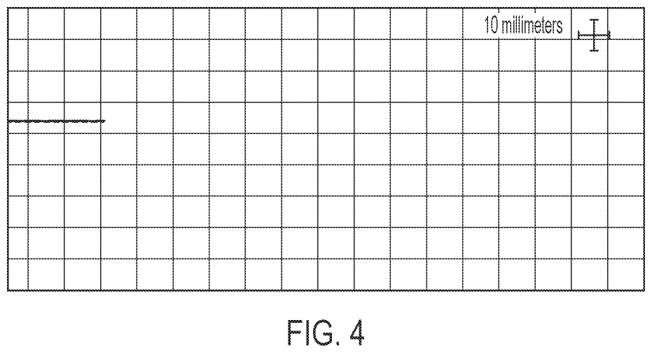

### DETAILED DESCRIPTION

Recently, research efforts related to hemostatic materials have focused on the use of bioactive agents, specifically hemostatic agents. However, many current formulations and devices still utilize materials derived from animal products.

### Definitions

“Administration,” or “to administer” means the step of giving (i.e. administering) a hemostatic device, material or agent to a subject. The materials disclosed herein can be administered via a number of appropriate routes.

“Hemostatic agent” means an agent that can initiate and stabilize blood clot growth during bleeding, including biologics such as thrombin, small molecules such as tranexamic acid (TXA), polymers such as feracrylum, peptides such as Thrombin Receptor Activating Peptides (TRAPs), polysulfonic acid polymers, sulfated icodextrin, sulfated carbohydrates, and inorganic materials such as kaolin.

“Hemostatic material” means a material comprising a hemostatic agent in a form suitable for application to a patient.

“Patient” means a human or non-human subject receiving medical or veterinary care.

“Pharmaceutical composition” means a formulation including an active ingredient. The word “formulation” means that there is at least one additional ingredient (such as, for example and not limited to, an albumin [such as a human serum albumin or a recombinant human albumin] and/or sodium chloride) in the pharmaceutical composition in addition to an active ingredient. A pharmaceutical composition is therefore a formulation which is suitable for diagnostic, therapeutic or cosmetic administration to a subject, such as a human patient. The pharmaceutical composition can be: in a lyophilized or vacuum dried condition, a solution formed after reconstitution of the lyophilized or vacuum dried pharmaceutical composition with saline or water, for example, or; as a solution that does not require reconstitution. As stated, a pharmaceutical composition can be liquid, semi-solid, or solid. A pharmaceutical composition can be animal-protein free.

“Therapeutically effective amount” means the level, amount or concentration of an agent, material, or composition needed to achieve a treatment goal.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

The instant disclosure provides hemostatic materials comprising at least one hemostatic agent and at least one substrate.

Hemostatic Agents

Disclosed hemostatic materials comprise hemostatic agents. For example, in embodiments, the hemostatic agent can comprise small molecules such as tranexamic acid (TXA), feracrylum, peptides such as Thrombin Receptor Activating Peptides (TRAPs), polysulfonic acid polymers, sulfated icodextrin, sulfated carbohydrates, and inorganic materials such as kaolin.

In embodiments the hemostatic agent can comprise a synthetic agent, such as a biocompatible polymer of an acid, for example a polyacrylic acid. In embodiments the hemostatic agent can comprise a polyacrylic polymer, for example a ferric salt of a polyacrylic polymer, a salt thereof, or an incomplete salt thereof.

In embodiments, the hemostatic agent can comprise feracrylum:

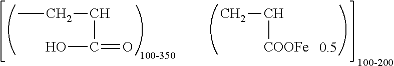

In disclosed embodiments, the hemostatic agent, for example feracrylum, is present in the hemostatic material at a weight or volume percentage of, for example, at least 1%, at least 2%, at least 3%, at least 4%, at least 5%, at least 6%, at least 7%, at least 8%, at least 9%, at least 10%, or more.

Substrates

Disclosed embodiments comprise carrier substrates such as granules, for example granules comprising cross-linked hydrogels comprising at least one biologic or non-biologic polymer, for example proteins, polysaccharides, and synthetic polymers.

In embodiments the substrate polymer is biodegradable. Biodegradable polymers release contained drugs as the matrix is consumed or biodegraded during therapy. The polymer is usually selected to breakdown into subunits which are biocompatible with the surrounding tissue. The persistence of a biodegradable polymer in vivo depends on its molecular weight and degree of cross-linking, the higher the molecular weights and degrees of cross-linking resulting in a longer life. Common biodegradable, polymers include polylactic acid (PLA, also referred to as polylactide), polyglycolic acid (PGA), copolymers of PLA and PGA, polyamides, and copolymers of polyamides and polyesters.

In various embodiments, the substrate material comprises a recombinant polymer. In particular, the recombinant polymer can be a recombinant human collagen, such as, for example, recombinant human collagen type I, recombinant human collagen type III, or a combination thereof. In one embodiment, the substrate material comprises recombinant human collagen type III. In another embodiment, the substrate material comprises recombinant human collagen type I. For example, the recombinant human gelatin can be derived from recombinant human collagen type III. In yet another embodiment, the substrate material comprises recombinant gelatin derived from recombinant human collagen type I. In further embodiments, the substrate material comprises recombinant gelatin produced directly by expression of encoding polynucleotide.

The polysaccharide used as a biocompatible substrate material in disclosed embodiments can comprise, for example, cellulose, alkyl cellulose, methylcellulose, alkylhydroxyalkyl cellulose, hydroxyalkyl cellulose, cellulose sulfate, salts of carboxymethyl cellulose, carboxymethyl cellulose, carboxyethyl cellulose, chitin, carboxymethyl chitin, hyaluronic acid, salts of hyaluronic acid, alginate, alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, chitosan, heparin, heparin sulfate, heparan, heparan sulfate, dermatan sulfate, keratan sulfate, carrageenans, chitosan, starch, amylose, amylopectin, poly-N-glucosamine, polymannuronic acid, polyglucuronic acid, polyguluronic acid, derivatives of said polysaccharides, or combinations thereof.

The present biocompatible substrate material can also be based on a synthetic polymer. The synthetic absorbable polymer can be an aliphatic polyester polymer, an aliphatic polyester copolymer, or combinations thereof.

In embodiments, the polymer is capable of being cross-linked and hydrated to form a hydrogel. Exemplary polymers include proteins selected from gelatin, collagen (e.g. soluble collagen), albumin, hemoglobin, fibrinogen, fibrin, fibronectin, elastin, keratin, laminin, casein and derivatives and combinations thereof. Alternatively, the polymer may comprise a polysaccharide, such as a glycosaminoglycan (e.g., hyaluronic acid or chondroitin sulfate), a starch derivative, a cellulose derivative, a hemicellulose derivative, xylan, agarose, alginate, chitosan, and combinations thereof. As a further alternative, the polymer may comprise a non-biologic hydrogel-forming polymer, such as polyacrylates, polymethacrylates, polyacrylamides, polyvinyl polymers, polylactide-glycolides, polycaprolactones, polyoxyethylenes, and derivatives and combinations thereof.

Cross-linking of the polymer may be achieved in any conventional manner. For example, in the case of proteins, cross-linking may be achieved using a suitable cross-linking agent, such as an aldehyde, sodium periodate, epoxy compounds, and the like. Alternatively, cross-linking may be induced by exposure to radiation, such as v-radiation or electron beam radiation. Polysaccharides and non-biologic polymers may also be cross-linked using suitable cross-linking agents and radiation. Additionally, non-biologic polymers may be synthesized as cross-linked polymers and copolymers. For example; reactions between mono- and poly-unsaturated monomers can result in synthetic polymers having controlled degrees of cross-linking. Typically; the polymer molecules will each have a molecular weight in the range from 20 kD to 200 kD, and will have at least one link to another polymer molecule in the network, often having from 1 to 5 links, where the actual level of cross-linking is selected in part to provide a desired rate of biodegradability in the ranges set forth below. Exemplary methods for producing molecular cross-linked gelatins are as follows.

Gelatin is obtained and placed in an aqueous buffer to form a non-cross-linked hydrogel; typically having a solids content from 1% to 70% w/w, usually from 3% to 10% by weight. The gelatin is then cross-linked, typically by exposure to either glutaraldehyde (e.g. 0.01% to 0.05% w/w, overnight at 0° C. to 15° C. in aqueous buffer), sodium periodate (e.g. 0.05 M, held at 0° C. to 15° C. for 48 hours) or 1-ethyl-3-(3-dimethylaminopropyl) carbodiimide (“EDC”) (e.g., 0.5% to 1.5% w/w, overnight at room temperature), or by exposure to about 0.3 to 3 megarads of gamma or electron beam radiation.

Alternatively, gelatin particles can be suspended in an alcohol, preferably methyl alcohol or ethyl alcohol, at a solids content of 1% to 70% by w/w, usually 3% to 10% by weight; and cross-linked by exposure to a cross-linking agent; typically glutaraldehyde (e.g., 0.01% to 0.1% w/w, overnight at room temperature). In the case of aldehydes, the pH should be held from about 6 to 11, preferably from 7 to 10. When cross-linking with glutaraldehyde, the cross-links are formed via Schiff bases which may be stabilized by subsequent reduction, e.g. by treatment with sodium borohydride. After cross-linking, the resulting granules may be washed in water and optionally rinsed in an alcohol, dried and resuspended to a desired degree of hydration in an aqueous medium having a desired buffer and pH. The resulting hydrogels may then be loaded into the applicators of the present invention; as described in more detail hereinafter. Alternatively, the hydrogels may be mechanically disrupted prior to or after cross-linking, also as described in more detail hereinafter. In embodiments, genipin can be employed as a cross-linker.

The extent of cross-linking of the polymer has an effect on several functional properties of the hydrogel including extrudability; adsorptiveness of surrounding biological fluids, cohesiveness, ability to fill space, swelling ability and ability to adhere to the tissue site. The extent of cross-linking of the polymeric hydrogel composition may be controlled by adjusting the concentration of cross-linking agent, controlling exposure to cross-linking radiation, changing the relative amounts of mono- and poly-unsaturated monomers, varying reaction conditions, and the like. Typically, the degree of cross-linking is controlled by adjusting the concentration of cross-linking agent.

The hydrogel compositions of the present invention will typically have a solids content in the range from 1% by weight to 70% w/w. Optionally, the compositions may comprise at least one plasticizer as described in more detail below. Suitable plasticizers include polyethylene glycols, sorbitol, glycerol, and the like.

The equilibrium swell of the cross-linked polymers of the present indisclosure may range from 400% to 5000%, 400% to 3000%, 400% to 2000%, usually ranging from 400% to 1300%, preferably being from 500% to 1100%, depending on its intended use. Such equilibrium swell may be controlled by varying the degree of cross-linking, which in turn is achieved by varying the cross-linking conditions, such as the type of cross-linking method, duration of exposure of a cross-linking agent, concentration of a cross-linking agent, cross-linking temperature, and the like.

Exposure to radiation, such as γ-radiation, may also be carried out in order to sterilize the compositions before or after packaging. When the compositions are composed of radiation-sensitive materials, it will be necessary to protect the compositions from the undesirable effects of sterilizing radiation. For example, in some cases, it will be desirable to add a stabilizer, such as ascorbic acid, in order to inhibit degradation and/or further excessive cross-linking of the materials by free radical mechanisms.

Methods of Manufacture

In embodiments, the feracrylum hemostatic agent can be prepared by polymerizing acrylic acid in an aqueous solution in the presence of this reduction-oxidation system: FeSO₄(NH₄)₂SO₄.6H₂O/K₂S₂O₈.

In embodiments, the polymerization can be carried out at a temperature of, for example, 25° C.

In embodiments, FeSO₄(NH₄)₂SO₄.6H₂O is in an amount of, for example, between 0.8 and 2.2 percent (w/w). The concentration of acrylic acid in the solution can be, for example, no more than 20 percent by volume, such 5%, 10%, 15%, 20%, or the like. The resulting viscous red mass is dissolved in water to a concentration of, for example, 3 to 4%.

In order to remove the initiator and unreacted acrylic acid, the incomplete ferric salt of polyacrylic acid (the feracrylum) is either re-precipitated from the aqueous solution with a saturated aqueous solution of sodium chloride with a subsequent dialysis of the polymer solution aimed to remove the captured NaCl, or is passed through a strong-base anion-exchange resin.

The purified polymer solution can be diluted, for example, to a concentration of 1 to 2 percent (in this form it is ready for use), or dried at a temperature of 50° C. under atmospheric pressure. The yield of feracrylum is 85-95 percent of the theoretical; this salt is a glasslike brittle mass of a orange-brownish color. It is readily soluble in water, but is insoluble in alcohols, dioxane, aliphatic hydrocarbons and their chlorine derivatives.

The molecular weight of the preparation's active principle is 7×10⁵ to 5×10⁶. The iron content in the salt is, in embodiments, 0.1 to 0.3% w/w.

Commercial Products/Kits

The present hemostatic materials can be finished as a commercial product by the usual steps performed in the present field, for example by appropriate sterilization and packaging steps. For example, the present material may be treated by UV/vis irradiation (200-500 nm), for example using photo-initiators with different absorption wavelengths (e.g. Irgacure 184, 2959), preferably water-soluble initiators (Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g. by the addition of specific product information leaflets) into suitable containers (boxes, etc.).

According to further embodiments, the hemostatic material can also be provided in kit form combined with other components necessary for administration of the material to the patient. For example, if the substrate material is provided in flowable dry form (e.g. as granules or as a powder) or as a flowable paste, it is preferred to provide such material with a liquid component comprising the hemostatic agent which can be added shortly before administration to the patient. Buffer components such as phosphate, carbonate, TRIS, etc., divalent metal ions, preferably Cat ions, or other functional components (if not already present on or in the substrate), such as anti-bacterial agents, immunosuppressive agents, anti-inflammatory agents, anti-fibrinolytic agents, such as aprotinin or ECEA, growth factors, vitamins, cells, etc. The kit may further contain means for administering or preparing administering the hemostatic material, such as syringes, tubes, catheters, forceps, scissors, sterilizing pads or lotions, etc.

Disclosed kits, such as for use in surgery and/or in the treatment of injuries and/or wounds, can comprise a disclosed hemostatic material and at least one administration device, for example a buffer, a syringe, a tube, a catheter, forceps, scissors, gauze, a sterilizing pad or lotion.

In embodiments, the buffer solution further comprises an anti-bacterial agent, immunosuppressive agent, anti-inflammatory agent, anti-fibrinolytic agent, especially aprotinin or ECEA, growth factor, vitamin, cell, or mixtures thereof. Alternatively, the kit can also further comprise an anti-bacterial agent, immunosuppressive agent, anti-inflammatory agent, anti-fibrinolytic agent, especially aprotinin or ECEA, growth factor, vitamin, cell, or mixtures thereof.

The kits are designed in various forms based on the specific deficiencies they are designed to treat.

Methods of Use

Methods of use of disclosed embodiments can comprise reconstituting the substrate, for example the cross-linked gelatin granules, with a solution containing a hemostatic agent, for example feracrylum, followed by application to a site where bleeding is desired to be reduced. For example, disclosed methods comprise application of disclosed embodiments to a site where bleeding is desired to be reduced, such as a site of injury or surgical procedure. These methods are further described in the following Examples.

Disclosed methods also comprise application of a hemostatic material to a site where microbial proliferation is desired to be reduced. For example, feracrylum exhibits an antimicrobial effect equivalent to that displayed by providone-iodine.

### EXAMPLES

The following non-limiting Examples are provided for illustrative purposes only in order to facilitate a more complete understanding of representative embodiments. This example should not be construed to limit any of the embodiments described in the present specification.

### Example 1

### Production of Feracrylum

Freshly distilled acrylic acid (28.8 g (0.4 mole)) is dissolved in 120 ml of distilled water. 1.7 g of K₂S₂O₈ dissolved in 20 mL of water is added to the solution thus obtained. The reaction mixture is then intensively stirred, as a solution of 0.24 g of FeSO₄(NH₄)₂SO₄.6H₂O in 2 ml of water is added thereto. The transparent viscous red mass thus produced is dissolved in 1 L of water. In order to remove the unreacted acrylic acid, the incomplete ferric salt of polyacrylic acid (the polymer) is re-precipitated two or three times from an aqueous solution, using a saturated aqueous solution of sodium chloride. The polymer is then again dissolved in water and dialyzed to remove the captured NaCl. The purified solution is dried at 50° C. Instead of salting out, the solution may be passed through an anion exchanger.

The iron content in the polymer thus produced is 0.11 percent by weight.

### Example 2

### Production of Feracrylum

Freshly distilled acrylic acid (28.8 g (0.4 mole)) is dissolved in 120 ml of distilled water. 1.7 g of K₂₅₂₀₈ dissolved in 20 ml of water is then added to the solution. The reaction mixture is intensively stirred, as a solution of 0.43 g of FeSO₄(NH₄)₂ SO₄.6H₂O is added thereto. After this stage, the process is carried out as in Example 1.

The iron content in the polymer thus produced is 0.2 percent by weight.

### Example 3

### Production of Feracrylum

Potassium persulphate (0.039 mole) is taken in a vessel containing 14.3 L of distilled water and stirred for 3 minutes. 26.08 mole of acrylic acid solution is added which is previously dissolved in 1.2 L of distilled water. This is further mixed with 0.0592 mole of ammonium ferrous sulphate dissolved in water. This is mixed thoroughly under continuous stirring for 3 to 4 hours. The mixture is diluted to 25 L and the whole mass is cooled to room temperature and kept for 2 hours. Resin is then added to remove impurities the mixture stirred for 30 minutes, filtered and evaporated under vacuum at 50° C. to 60° C. using rotary evaporator. The evaporated product is passed through a micronizer, which yields fine shining peach colored crystals. These crystals have the characteristics of rapid solubility and meet the general pharmaceutical specifications.

The feracrylum has the following specifications:
a. Water (by Karl Fischer) 1%, max;
b. Color Density of 1% aqueous solution at 420 nm in 1 cm cell 0.1, max;
c. Bulk density (g/mL) min. 0.6, max. 0.85;
d. Particle size Av. particle size 500 micron.

The feracrylum thus prepared readily dissolves in water at 25° C., is easily filterable and can also be easily sterilized.

### Example 4

### Production of Hemostatic Material

Feracrylum as prepared in Examples 1-3 is added to a substrate comprising cross-linked gelatin granules to form a hemostatic material.

### Example 5

### Treatment of Injury

An automobile accident victim sustains traumatic injuries to the abdomen. To stop blood loss, a disclosed hemostatic material is applied to the injury site. Blood loss is reduced within minutes.

### Example 6

### Treatment of Surgical Incision

To stop blood loss, a disclosed hemostatic material is applied to the site of a surgical incision. Blood loss is reduced within minutes, the hemostatic material also provides an antimicrobial effect.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A hemostatic material comprising a gelatin matrix and a synthetically-derived hemostatic agent.

2. The hemostatic material of claim 1, wherein said gelatin matrix comprises a cross-linked gelatin.

3. The hemostatic material of claim 2, wherein said cross-linked gelatin comprises gelatin granules.

4. The hemostatic material of claim 3, wherein said synthetically-derived hemostatic agent comprises a biodegradable polymer.

5. The hemostatic material of claim 4, wherein said biodegradable polymer comprises feracrylum.

6. The hemostatic material of claim 5, wherein said hemostatic material is flowable.

7. A kit for use in establishing local hemostasis, comprising a gelatin matrix and a synthetically-derived hemostatic agent, wherein said kit further comprises at least one administration device, buffer solution, diluent, syringe, tube, catheter, forceps, scissors, sterilizing pad or lotion.

8. The kit of claim 7, wherein said gelatin matrix comprises a cross-linked gelatin.

9. The kit of claim 8, wherein said cross-linked gelatin comprises gelatin granules.

10. The kit of claim 9, wherein said synthetically-derived hemostatic agent comprises a biodegradable polymer.

11. The kit of claim 10, wherein said biodegradable polymer comprises feracrylum.

12. A method of controlling bleeding comprising applying a hemostatic material to a treatment site, said hemostatic material comprising a gelatin matrix and a synthetically-derived hemostatic agent.

13. The method of claim 12, wherein said gelatin matrix comprises a cross-linked gelatin.

14. The method of claim 13, wherein said cross-linked gelatin comprises gelatin granules.

15. The method of claim 14, wherein said synthetically-derived hemostatic agent comprises a biodegradable polymer.

16. The method of claim 15, wherein said biodegradable polymer comprises feracrylum.

17. The method of claim 16, wherein said hemostatic material is flowable.
